(19) United States
(12) Patent Application Publication
(10) Pub. No.: US 2004/0253326 A1
(43) Pub. Date: Dec. 16, 2004

(54) Composition for increasing levels of hormones and a method for preparation of said composition

(76) Inventor: Charles A. Mesko, Millersport, OH (US)

Correspondence Address: WOOD, HERRON & EVANS, LLP, 2700 CAREW TOWER, 441 VINE STREET, CINCINNATI, OH, 45202, US

(21) Appl. No.: 10/790,417

(22) Filed: Mar. 1, 2004

Related U.S. Application Data
(63) Continuation-in-part of application No. 10/374,594, filed on Feb. 25, 2003.

(51) Int. Cl. A61K 35/78; A61K 31/56

(52) U.S. Cl. 424/725; 514/171

(57) ABSTRACT

The present invention includes a pharmacologically acceptable composition for ingestion by a mammal, having a first ingredient including a hormone or a substance which stimulates production of a hormone, such as testosterone or growth hormone. The composition also may include a second ingredient which stimulates the production of cyclic GMP. The second ingredient may also be Morinda citrifolia or an extract thereof. The composition of the present invention increases levels of a hormone with a body.

### CROSS-REFERENCE TO RELATED APPLICATIONS

This application is a continuation-in-part of and claims priority to U.S. patent application Ser. No. 10/374,594, entitled “Composition for Increasing Levels of Hormones and a Method for Preparation of said Composition,” filed Feb. 25, 2003 by Charles A. Mesko, which is hereby incorporated by reference herein in its entirety.

### FIELD OF THE INVENTION

The present invention relates generally to a composition for varying the levels of hormones in a mammal and specifically to a composition for introduction into the human system, that results in increased levels of testosterone and/or human growth hormone.

### BACKGROUND OF THE INVENTION

Testosterone is a physiological substance produced by the human body and is responsible for normal growth and development of male sex organs and maintenance of secondary sex characteristics. It is the primary androgenic hormone, and its production and secretion by the testes are the end product of a series of hormonal interactions referred to as the hypothalamic-pituitary-gonadal axis. Gonadotropin-releasing hormone (GnRH) is secreted by the hypothalamus to the pituitary gland in carefully timed pulses. This triggers the pulsatile secretion of luteinizing hormone (LH) and follicle-stimulating hormone (FSH) by the anterior pituitary. Luteinizing hormone regulates the production and secretion of testosterone by the Leydig cells of the testes and FSH stimulates spermatogenesis.

When the testes fail to produce normal levels of testosterone, testosterone deficiency results. Hypergonadotropic hypergonadism is caused by primary testicular failure. Testosterone levels are low and pituitary gonadotropins are elevated. In secondary, more hypogonadotropic hypergonadism, there is inadequate secretion of pituitary gonadotropins. In addition to a low testosterone level, LH and FSH levels are low or low-normal. While prepubertal hypergonadism is generally characterized by infantile genitalia and lack of virilization, the development of hypogonadism after puberty frequently results in complaints such as diminished libido, erectile dysfunction, infertility, gynecomastia, impaired masculinization, changes in body composition, reductions in body and facial hair, and osteoporosis. In addition to these complaints, mood inventory scores indicate that hypogonadal men report levels of anger, confusion, depression, and fatigue that are significantly higher than those reported by men with normal testosterone levels.

Testosterone levels begin to decline in men at about age 25 and decrease steadily with age. Testosterone levels may drop 2% yearly. Deficiency symptoms include decreased sex drive, loss of muscle mass and strength, decreased bone density, lessened self esteem, and increased body fat. Many men's testosterone levels may become severely deficient between 50-60 years of age.

Human growth hormone (somatropin) is a physiological substance produced in the anterior lobe of the pituitary gland of the human system. It is the most abundant hormone produced by the anterior pituitary lobe, accounting for as much as eight to ten percent of the dry weight of the gland. The physiological effects of human growth hormone are macroscopic, extending beyond cellular, chemically mediated events. One example of this is the effect of human growth hormone on whole body growth. Growth hormone effects an increase in tissue and organ weight, which results from an increase in mitosis, cellular hypertrophy, hyperplasia and cellular water. Growth hormone also stimulates the increased uptake of amino acids into cells, resulting in protein synthesis. Growth hormone is responsible for proper growth and development until adulthood and then regulates nearly every organ in the body. Growth hormone also has prosexual effects, such as enhanced sexual performance and a decrease in the incidence of impotence in males. Studies have suggested that an estimated 70%-80% of cases of erectile dysfunction may be caused by increased levels of prolactin, another hormone released by the pituitary gland. Growth hormone release from the pituitary gland can be associated with a decrease in the release of prolactin.

Growth hormone is synthesized in the acidophilic somatotropes, which are specialized cells located in the anterior of the pituitary gland. Secretion of the growth hormone by the pituitary gland is episodic. The anterior pituitary operates in conjunction with the hypothalamus and adrenal glands as an integrated unit. The large array of physiological factors that effect growth hormone secretion act on the pituitary gland through the hypothalamus. The hypothalamus controls this secretion by causing the release of either growth hormone release stimulating or inhibiting factors. These factors result in the release or retention of human growth hormone by the pituitary gland.

Human growth hormone was first discovered by researchers in the 1920s, and due to its effects on whole body growth, was considered to be a promising therapeutic agent. In 1958, growth hormone extracted from the pituitary glands of cadavers was injected into a growth stunted child at the New England Medical Center in Boston, Mass. As a result, the child grew taller and popularity for the use of growth hormone in medical applications grew.

However, one major drawback of the natural growth hormone, is that heat destroys the pituitary extract. Thus, it cannot be pasteurized to eliminate the possibility of disease transmission. Therefore, before the use of growth hormone as a therapeutic agent could become widespread, it either had to be sterilized or a synthetic hormone had to be produced to reduce the potential for transmission of disease.

In 1986, the Eli Lilly Company developed “Humatrope”, a manmade growth hormone identical in structure to the actual human growth hormone. Soon after, in 1990, the New England Journal of Medicine released the results of a six month clinical study on human growth hormone administered to a group of men aged 61 to 81. Without exercise, these men lost body fat and wrinkles and gained lean muscle, along with a thickening of the skin and a regeneration of failing liver tissue.

Thus, testosterone and human growth hormone may be used for therapeutic benefits in persons who have deficient hormone levels. Additionally, testosterone and human growth hormone may be used for cosmetic benefits. As a result, there has been an increased demand for testosterone and/or human growth hormone treatments. However, a number of problems are associated with such use. First, cost is a factor. For example, increased levels of testosterone or human growth hormone may be provided through the injection of the particular hormone (natural or synthetic) from a foreign source directly into the human subject. These treatments however, may come at an increased cost, such that they may be prohibitively expensive. Additionally, when the delivery method is via injection, the injections have to be performed in a medical environment under a physician's supervision. This requirement not only adds to the cost of the treatment, but is also inconvenient to the patient's schedule.

Second, increasing levels of a foreign hormone, such as testosterone or human growth hormone, within a subject also may result in negative side effects. For example, the addition of any testosterone to the human system may also negatively affect the body's natural production of testosterone by curtailing the production of that natural testosterone. Thus, any addition of testosterone to the human system, whether natural or synthetic, will impact the body's negative feedback loop as it relates to the production of testosterone, thereby disrupting the balance of testosterone levels achieved by the operation of the hypothalamic-pituitary-gonadal axis. As a result, the testes may slow down or even cease production and release of natural testosterone, and the normal function of the hypothalamic-pituitary-gonadal axis of the subject is disrupted. Additionally, another problem with traditional testosterone supplement preparations results from the body's own natural defense mechanism to convert higher levels of testosterone into estrogen. Thus, when testosterone levels increase, the body responds by aromatizing the excess testosterone into estrogen. This may result in artificially high levels of estrogen in a subject.

Another problem that results from decreased levels of testosterone, as mentioned above, is the deterioration of prosexual characteristics in the body. Certain of these characteristics involve a deterioration of the physical manifestations associated with libido. One example of this is the lack of vasodilation of penile arteries and a resultant weakening of erections in males. Unfortunately, the testosterone treatments described above do not rectify this situation.

In the case of growth hormone, in response to the injection of a foreign growth hormone (natural or synthetic), the hypothalamus triggers elevated levels of somatostatin, a growth hormone release inhibitor, which then prompts the pituitary gland of the subject individual to curb the release of its naturally produced growth hormone. Of course, it is undesirable to inhibit the natural release of the growth hormone from the pituitary gland in favor of the synthetic hormone. This is because the release of natural growth hormone from the pituitary gland is controlled by negative feedback involving growth hormone releasing and release inhibiting factors. If growth hormone levels are low, the pituitary gland is stimulated by releasing factors in the hypothalamus to increase the release of natural growth hormone. If growth hormone levels are high, the pituitary gland is inhibited from releasing natural growth hormone by the release inhibiting factor, somatostatin, from the hypothalamus. Thus any addition of growth hormone to the human system, whether natural or synthetic, will impact this negative feedback loop, thereby disrupting the balance of growth hormone levels achieved by the tandem operation of hypothalamus and pituitary gland. As a result, the pituitary gland may slow down or even cease production and release of the natural growth hormone, and the normal function of the pituitary gland of the subject is disrupted.

Due to the aforementioned drawbacks with current procedures for increasing hormone levels, such as testosterone or growth hormone, within the body, it is desirable to increase hormone levels in the human system without negatively affecting the natural production and release of those hormones by the body. Thus, it is desirable to increase testosterone levels in the human system without negatively affecting the natural production and release of testosterone by the testes. Likewise, it is desirable to increase growth hormone levels in the human system without negatively affecting the natural production of growth hormone by the pituitary gland. It is also desirable to increase hormone levels in a manner which does not require physician supervision. It is further desirable that any such composition or method to achieve such objectives be available at a low cost. It would be further desirable that such a low cost composition or method enhance prosexual characteristics.

### SUMMARY OF THE INVENTION

The present invention solves the above problems and addresses the above objectives by increasing levels of hormones, such as testosterone or growth hormone, within the human body. In particular, in a first embodiment, the present invention includes a pharmacologically acceptable composition for administration to a mammal which increases the level of testosterone in the body. This composition includes a first ingredient being either testosterone or a substance for stimulating production of testosterone and a second ingredient for stimulating the production of guanosine 3′,5′-(cyclic) phosphate (cyclic GMP), which is a phosphate that causes the relaxation of smooth muscle tissue. The composition may also include a third ingredient for stimulating an increase in blood flow.

In particular, when the composition includes a substance to stimulate the production of testosterone, the first ingredient may be a substance known to have an activity to affect levels of luteinizing hormone within the human system. In particular, the first ingredient may be an herb or herbal extract which has activity to affect luteinizing hormone. This herb or herbal extract may include Eurycoma longifolia jack. Alternatively, the first ingredient may include Tribulus L. Terrestris.

In addition to the first ingredient, the second and third ingredients may operate to enhance prosexual characteristics by enhancing physical manifestations of the increased levels of testosterone. In doing so, the ingredients of the composition may synergistically accentuate the body's increased production of testosterone. To accomplish this, the second ingredient which stimulates production of cyclic GMP may include a coumarin. The coumarin of the second ingredient may stimulate production of cyclic GMP by having an activity to stimulate the production of nitric oxide. An excess of nitric oxide in the human system leads to the production of cyclic GMP. In particular, the coumarin may be osthole. In particular, the second ingredient which may include this coumarin may be Cnidium monnier. Additionally, the second ingredient may have a further activity to inhibit the activity of at least one enzyme. In particular, the enzyme to be inhibited is a phophodiesterase, such as phosphodiesterase-5 (PDE-5). PDE-5 binds to and digests cyclic GMP.

Finally, the third ingredient, which may stimulate an increase in blood flow, can be Epimedium sagittatum. An increased blood flow throughout the body will also result in an increased blood flow in penile arteries, which assists in sustained erections in males. In synergistic fashion, the cyclic GMP simulated by the second ingredient acts to relax smooth muscle tissue. This results in vasodilation to accommodate the increased blood flow resulting from the third ingredient of the composition.

In a second embodiment, the present invention includes a pharmacologically acceptable composition for administration to a mammal which increases levels of growth hormone within a body. The composition includes a first ingredient, being growth hormone or a substance to potentiate a body's own production of growth hormone. The composition may also include a second ingredient including an herb or herbal extract to enhance the effects of the first ingredient. In particular, the second ingredient can be Morinda citrifolia (Noni) or an herbal extract of Morinda citrifolia.

In particular, the first ingredient includes an herb or herbal extract which may include either an element for synthesizing a catecholamine, or an element having an active component comprising a luteinizing agent. Alternatively, the composition may include a first herbal extract including an element for synthesizing a catecholamine, and a second herbal extract having an active component comprising a luteinizing agent.

There are several factors which may stimulate growth hormone release by the pituitary. One group of releasing factors includes catecholamines, which are amine derivatives of dihydroxybenzene (or catechol), including norepinephrine, epinephrine and dopamine. Upon introduction to the body, the composition of the present invention may act to affect the production of dopamine within the human system.

The presence of dopamine in the human system stimulates the release of growth hormone by the pituitary gland. Thus, levels of dopamine may be increased by a first herb or herbal extract of the composition of the present invention which contains dihydroxyphenylalanine (L-dopa). Once introduced into the human system, L-dopa converts to dopamine and stimulates an increase in serum concentration levels of growth hormone. By using dopamine to stimulate the release of the human system's own naturally-occurring growth hormone, the composition of the present invention avoids disrupting the normal function of the pituitary gland through the use of a foreign growth hormone which is a problem with prior art methods and compositions. L-dopa is also an effective inhibitor of the release of the hormone prolactin by the pituitary gland. Increased levels of prolactin in the human system are responsible for an estimated 70% -80% of erection failures in males. Therefore, inhibiting prolactin release in accordance with the principles of the invention will limit erection failures in males.

As described above, the composition of the second embodiment of the present invention may include first and second herbs or herbal extracts. In this embodiment, the second herb or herbal extract may also prevent the L-dopa of the first herb or herbal extract from breaking down in the human system and also helps to maintain the presence of dopamine in the human system for an extended period of time. Further, in this embodiment of the present invention, the combination of the first and second herbs or herbal extracts of the present invention helps to block the release of somatostatin. As described above, somatostatin is a factor that inhibits the release of growth hormone. As the concentration of growth hormone in the human system rises, the hypothalamus releases somatostatin to control growth hormone levels, as part of the negative feedback loop discussed above. Any release of somatostatin operates counter to the benefits of higher concentrations of growth hormone that the present invention provides. Thus, by blocking the release of somatostatin, the combination of herbs or herbal extracts of the present invention maximizes levels of growth hormone in the human system.

In another aspect, the composition of the present invention is self-administrable and thus available at lowered cost. The composition may be provided in capsule, pill, or tablet form. Alternatively, the composition may be provided in a form suitable for topical administration. The composition may thus proceed from a first site external to the body to a second site internal to the body by being absorbed transdermally. In this manner the composition addresses several drawbacks associated with prior art treatments involving the injection of hormones, such as testosterone and growth hormone. For example, by eliminating the need for injection treatments, the present invention eliminates the need for physician supervision. This results in a reduction of inconvenience for the patient who, in receiving injections, had to manipulate schedules to include a doctor's appointment and had to endure what many patients consider to be discomforting: a needle injection. By providing the composition of the present invention in a form suitable for topical administration, the high costs of treatment associated with injection treatments are likewise reduced.

The present invention also includes a method of preparing a pharmacologically acceptable composition for topical administration to a mammal. This method includes providing a first ingredient being a hormone or an element for potentiating a hormone. This hormone may be testosterone, or alternatively may be growth hormone. The first ingredient may be provided in homeopathic form. The first ingredient may be combined with a plurality of active homeopathic ingredients. These active homeopathic ingredients may be liquified or, alternatively, may be solid. The combination of homeopathic first ingredient and active homeopathic ingredients may then be encased in liposomes by processes well known to those having skill in the relevant art.

Thus, by this process, the ingredients of the present invention may be included in various compositions adapted to be topically applied to the skin in the form of moisturizers, creams, lotions, gels, ointments, emulsions, etc. This allows the ingredients to enter the body transdermally, thus eliminating the potential breakdown of the ingredients that occurs when ingested orally, and eliminating the injection of hormones, physician supervision, etc. and attendant high costs.

The present invention also provides a method of increasing the level of a particular hormone, such as testosterone or growth hormone, in a mammal by administering the composition transdermally through topical administration.

By providing a composition as described above, the present invention increases hormone levels, such as testosterone or growth hormone, in the human system without negatively affecting the natural production and release of such hormones by the body. The composition may also result in an enhancement of physical manifestations caused by an increased level of testosterone or growth hormone, and thus increased libido. The use of the composition of the present invention may be provided in capsule or other self-administrable form such that it does not require physician supervision and is available at low cost. The above and other objects and advantages of the present invention shall be made apparent from the following detailed description.

### DETAILED DESCRIPTION OF THE INVENTION

The present invention provides a composition that increases levels of hormones, such as testosterone and growth hormone, within the human body. The composition of the present invention may accomplish this by using natural or synthetic hormones, or by potentiating the body's own natural production of hormones, such as testosterone or growth hormone. In particular, in a first embodiment, the present invention provides a composition for increasing levels of testosterone. In a second embodiment, the present invention provides a composition for increasing levels of growth hormone. The present invention also results in enhancement of prosexual characteristics in the human system.

In a first embodiment, the present invention particularly comprises a synergistic blend of ingredients to produce a very potent and effective testosterone-potentiating composition. In this embodiment, the present invention includes a pharmacologically acceptable composition for administration to a mammal, having a first ingredient which may be testosterone or may stimulate production of testerone, and a second ingredient for stimulating the production of cyclic GMP. When the first ingredient includes a substance to stimulate production of testosterone, the first and second ingredients of the composition have a synergistic effect when combined that stimulates a body's own production of testosterone, and leads to enhancement of prosexual characteristics within the body. In particular, the composition increases libido along with enhancing physical manifestations of libido, including causing sustained penile erections in males. Additionally, an increase in testosterone levels results in an increase in sex drive. Increased levels of testosterone may also cause physical changes, such as increased muscle mass and strength, increased bone density, and decreased body fat. These physical changes and increased sex drive may be further supplemented by the other ingredients of the composition. For example, the composition may include a third ingredient for stimulating an increase in blood flow in the body.

When the first ingredient stimulates the production of testosterone, the first ingredient of the composition may particularly include a substance having an activity to affect luteinizing hormone within the human system. Such a substance is referred to as a luteinizing agent. The luteinizing agent may be a saponin and/or alkaloid. Such luteinizing agents are known to stimulate the hypothalamic-pituitary-gonadal axis to increase levels of natural testosterone. In particular, the presence of luteinizing agents may serve, like luteinizing hormones, to regulate the production and secretion of testosterone by the Leydig cells of the testes. More specifically, these luteinizing agents may stimulate the secretion of luteinizing hormone by the anterior pituitary, which increases the production and secretion of testosterone by the Leydig cells of the testes.

Thus, the first ingredient may be an herb or herbal extract which has activity to affect levels of luteinizing hormone, as described above. Suitable herbs or herbal extracts include Eurycoma longifolia jack and Tribulus L. Terrestris. Eurycoma longifolia jack is a testosterone booster that may have an activity to affect the production of luteinizing hormone to signal the hypothalamus and pituitary to naturally increase testosterone levels. The active Eurycoma longifolia jack is generally derived from the root of the plant. In alternate embodiments of the present composition, the Eurycoma longifolia jack may be present either in the form of a root powder (generally prepared by drying the root of the plant, followed by pulverizing the root) or in a root extract (wherein a more pure form of the Eurycoma longifolia jack is extracted from the root powder). Particularly, a root extract form of Eurycoma longifolia jack, including at least 40% glycosaponins and at least 20% glycoproteins, is used in the composition of the present invention. In one particular embodiment, Eurycoma longifolia jack may be present in the composition of the present invention in an amount of at least about 0.09 percent by volume. Eurycoma longifolia jack may be present in a range of about 50 mg to about 300 mg in a 4 oz. (118 ml) solution. In one particular embodiment, for example, 100 mg of Eurycoma longifolia jack may be present in 4 oz. (118 ml) solution. For administration to a mammal, Eurycoma longifolia jack may be provided in a dosage amount in a range of about 0.02 mg/kg to about 0.06 mg/kg. Frequency of administration of a dosage to a mammal can be twice per day. Thus, the amount of Eurycoma longifolia jack administered per day may be in a range of about 0.04 mg/kg/day to about 0.12 mg/kg/day.

Tribulus L. Terrestris is an Ayruvedic herb. The Tribulus L. Terrestris may be present in the composition as an herb or herbal extract. It is known to increase seminal fluid by sperm count and at the same time increase libido. Tribulus L. Terrestris has also been shown to increase the duration of penile erections in males. Tribulus L. Terrestris is known to stimulate the secretion of luteinizing hormones from the anterior pituitary gland, which triggers testosterone production. Tribulus L. Terrestris includes saponins of the furostanol type, including protodioscin. Particularly, an extract of Tribulus L. Terrestris containing at least 40% protodioscin can be used in the composition. Tribulus L. Terrestris can be present in the composition in an amount of at least 0.09 percent by volume. Tribulus L. Terrestris may be present in a range of about 50 mg to about 300 mg in a 4 oz. (1 18 ml) solution. In one particular embodiment, for example, 100 mg of Tribulus L. Terrestris may be present in 4 oz. (118 ml) solution. For administration to a mammal, Tribulus L. Terrestris may be provided in a dosage amount in a range of about 0.02 mg/kg to about 0.06 mg/kg. Frequency of administration of a dosage to a mammal can be twice per day. Thus, the amount of Tribulus L. Terrestris administered per day may be in a range of about 0.04 mg/kg/day to about 0.12 mg/kg/day.

It will be recognized that the first ingredient is not limited to herbs or herbal extracts. For example, the first ingredient can be the hormone itself, such as testosterone or growth hormone.

Although the first ingredient, as described above, may be present in non-homeopathic form, alternatively, the first ingredient may be provided in a homeopathic form. The approach of homeopathy is to trigger the body's rejuvenation with the smallest possible dosage that will stimulate a response in a patient. This results in products that do not produce side effects, will not react to allopathic formulations, and do not cause habitual use or dependency.

Thus, the first ingredient can be Eurycoma longifolia jack provided in homeopathic form. Alternatively, the first ingredient may be Tribulus L. Terrestris provided in homeopathic form. Still alternatively, the first ingredient can be testosterone or growth hormone provided in homeopathic form. When including homeopathic Eurycoma longifolia jack, the Eurycoma longifolia jack may be present in the composition in an amount sufficient to increase levels of testosterone within the human system. In various embodiments of the present invention, the Eurycoma longifolia jack may be present in different potencies. In one embodiment, Eurycoma longifolia jack may be provided in a potency of 10×. In an alternate embodiment, the potency may be 30×. In a still alternate embodiment, the potency may be 100×. As those of skill in the art will recognize, each homeopathic ingredient is first obtained as an “original tincture” which includes 10 grams of active ingredient, for example Eurycoma longifolia jack, dissolved in 90 grams of solvent (a 10% solution). Next, the solution is repeatedly diluted. The strength of the solution increases with the degree of dilution. This may be expressed in “X” (a decimal dilution) or “C” (a centesimal solution). Thus, 1× means a 1:10 dilution (one part of the original matter in nine parts solvent). Thus, a 10× potency is a 1:10 dilution repeated 10 times. A 30× potency is a 1:10 dilution repeated 30 times. And a 100× potency is a 1:10 dilution repeated 100 times. The resulting dilutions then are combined in a 4 oz. (118 ml) solution. A dosage unit from this solution is in the range of about 0.03 oz. (0.9 ml) to about 0.08 oz. (2.4 ml). Paricularly, a dosage unit of this solution may be about 0.07 oz. (2.1 ml). Such a dosage amount may be administered to a mammal, such as a human. Frequency of administration of a dosage to a mammal can be twice per day.

In addition to the first ingredient, the composition of the present invention includes a second ingredient to stimulate the synthesis of guanosine 3′,5′-(cyclic) phosphate (“cyclic GMP”). Cyclic GMP is a component of a signal transduction pathway, and ultimately affects relaxation of smooth muscle tissue and vasodilation. In this manner, which will be described in greater detail below, the second ingredient may work synergistically with the first ingredient to provide a composition to increase libido and expand penile arteries so that they may fill with blood.

Cyclic GMP is synthesized in response to elevated levels of nitric oxide. Nitric oxide stimulates enzymes, such as soluble guanylyl cyclase. Guanylyl cyclase then synthesizes cyclic GMP which is known to activate protein kinase G. Protein kinase G mediates vasodilation, that is, the increase in the diameter of the blood vessel and in consequence, the decrease in blood pressure. Thus, stimulation of the production of cyclic GMP by the second ingredient thus assists in vasodilation. This may lead to penile arteries filling with blood. It also operates in conjunction with the first ingredient to enhance sex drive and cause sustained erections in males. Cyclic GMP is degraded by a group of enzymes known as phophodiesterases (PDE). Degradation of cyclic GMP will reverse the relaxation of smooth muscle tissue.

The second ingredient may be an herb or herbal extract which includes a compound to cause increased levels of nitric oxide. Suitable ingredients include Cnidium monnier, which is an herb, and an extract of that herb. Cnidium monnier is a plant which contains several compounds, including coumarins, (such as osthole), imperatorin, glucides and hepatoprotective sesquiterpenes. Coumarins are known to stimulate the production of nitric oxide. The coumarin provided by the second ingredient may be osthole. Osthole particularly stimulates the production of nitric oxide, which leads to the production of cyclic GMP (cGMP). Cyclic GMP ultimately affects smooth muscle relaxation. This assists in dilation of blood vessels. Osthole further assists in dilation of blood vessels due to its calcium channel blocking properties. As a result of the smooth muscle relaxation and dilation of blood vessels, the second ingredient has an effect of allowing penile arteries to expand and fill with blood. In particular, in this embodiment, Cnidium monnier may be present in the composition in an amount of at least 0.09 percent by volume. Cnidium monnier may be present in a range of about 50 mg to about 300 mg in a 4 oz. (118 ml) solution. In a particular embodiment, 100 mg of Cnidium monner may be present in 4 oz. (118 ml) solution. For administration to a mammal, Cnidium monner may be provided in a dosage amount in a range of about 0.02 mg/kg to about 0.06 mg/kg. Frequency of administration of a dosage to a mammal can be twice per day. Thus, the amount of Cnidium monnier administered per day may be in a range of about 0.04 mg/kg/day to about 0.12 mg/kg/day. Cnidium monnier, also is known to inhibit PDE-5 which binds and digests cyclic GMP.

In addition to the hormone source and the cGMP source, the present invention may include a third ingredient which stimulates blood flow. The third ingredient may be an herb or herbal extract, such as Epimedium sagittatum. Epimedium sagittatum has androgen-like effects. Androgens are involved in increasing libido. Epimedium sagittatum lowers blood pressure by dilating blood vessels. In particular, the second ingredient stimulates cerebral and peripheral circulation; dilates coronary blood vessels; increases coronary blood flow by reducing vascular resistance; and cleanses the kidneys. Epimedium sagittatum includes a flavonoid known as icariin which increases testosterone levels. Epimedium sagittatum also results in a further increase of prosexual characteristics. In particular, a dilation of the blood vessels allows penile arteries to expand and fill with blood, leading to sustained erections in males. This effect is increased as a result of the increased levels of testosterone resulting from the first ingredient of the composition of the present invention. An extract of Epimedium sagittatum having at least 20 percent icariin can be used in the composition. Epimedium sagittatum can be present in the composition in an amount of at least 0.09 percent by volume. Epimedium sagittatum can be present in an amount of about 50 mg to about 300 mg per 4 oz. (118 ml) solution. In particular, 100 mg Epimedium sagittatum may be present in 4 oz (118 ml) solution. For administration to a mammal, Epimedium sagittatum may be provided in a dosage amount of about 0.02 mg/kg to about 0.06 mg/kg. Frequency of administration of a dosage to a mammal can be twice per day. Thus, the amount of Epimedium saggitatum administered per day may be in a range of about 0.04 mg/kg/day to about 0.12 mg/kg/day. In an alternate embodiment, Epimedium sagittatum may be present in homeopathic form.

Another major problem with traditional testosterone preparations, as described above, is the body's own natural defense mechanism to convert higher levels of testosterone into estrogen. When testosterone levels increase, the body responds by aromatizing the excess testosterone into estrogen. The composition of the present invention eliminates this problem, in one embodiment, by including an estrogen blocker in the formula of the composition. Suitable estrogen blockers for use in the composition include chrysin and diindolylmethane. Chrysin has its activity in filling estrogen receptor sites, thereby preventing estrogen production. The chrysin may be present in the composition in an amount of at least 0.09 percent by volume. Chrysin can be present in a range of about 50 mg to about 300 mg per 4 oz. (118 ml) solution. Particularly, 100 mg chrysin may be present in 4 oz. (118 ml) solution of the composition. For administration to a mammal, chrysin may be provided in a dosage amount of about 0.02 mg/kg to about 0.06 mg/kg. Frequency of administration of a dosage to a mammal can be twice per day. Thus, the amount of chrysin administered per day may be in a range of about 0.04 mg/kg/day to about 0.12 mg/kg/day.

Diindolylmethane is a cruciferous extract which fills carcinogenic estrogen receptor sites. Estrogen production decreases when estrogen receptor sites are occupied and cannot be used. This results in sustained increased testosterone levels. The diindolylmethane may be present in the composition of the present invention in an amount of at least 0.09 percent by volume. Diindolymethane can be present in a range of about 50 mg to about 100 mg per 4 oz. (1 18 ml) solution. Particularly, 100 mg diindolylmethane may be present in 4 oz. (118 ml) solution of the composition. For administration to a mammal, diindolylmethane may be provided in a dosage amount of about 0.02 mg/kg to about 0.06 mg/kg. Frequency of administration of a dosage to a mammal can be twice per day. Thus, the amount of diindolylmethane administered per day may be in a range of about 0.04 mg/kg/day to about 0.12 mg/kg/day.

The composition of the present invention may include a plurality of estrogen blockers. This plurality of estrogen blockers may include, but is not limited to, chrysin and diindolylmethane.

In addition to the above-listed ingredients which may be provided in the composition of the present invention, the composition may also include a plurality of additional active homeopathic ingredients. These active homeopathic ingredients may create a synergistic effect of the composition which is more effective than using each ingredient individually. Some of the active homeopathic ingredients may offer similar benefits that mimic a hormone, such as testosterone, and some may cause the body to produce more hormone, such as testosterone, by causing the stimulation of the body's own production of that hormone.

In the embodiment of the composition of the present invention including a first ingredient being a hormone or a compound which potentiates a hormone, such as testosterone, the plurality of active homeopathic ingredients may include one or more of adrenalinum, alfalfa, avena sativa, baryta carbonica, baryta iodata, baryta muriatica, calcarea carbonica, calcarea fluorica, calcarea phosphorica, lycopodium clavatum, and thuja occidentalis. Each of these active homeopathic ingredients may be present in concentrations of 1OX. Alternatively, each of the active homeopathic ingredients may be present in concentrations of 30×. Still alternatively, each of the active homeopathic ingredients may be present in concentrations of 100×. In one particular embodiment, potencies of 10×, 30×, and 100× of each ingredient are combined in a 4 oz. (118 ml) solution. A dosage unit from this solution is in the range of about 0.03 oz. (0.9 ml) to about 0.08 oz. (2.4 ml). Particularly, a dosage unit of this solution may be about 0.07 oz. (2.1 ml). Frequency of administration of a dosage to a mammal can be twice per day.

In addition to the ingredients listed above, the composition of the present invention may include a plurality of inactive ingredients. Such ingredients may include one or more of aloe barbadensis extract, polyacrylamide, C13-14 isoparaffin, indole-3-carbinol, laureth 7, lecithin, saw palmetto extract, and diazolidinyl urea. The composition may include each of the above listed inactive ingredients. These inactive ingredients may be present in equal amounts, such as at least 0.01 percent by volume each, or, alternatively may be present in varying amounts. For administration to a mammal, the dosage amount for each inactive ingredient may range from about 0.002 mg/kg to about 0.004 mg/kg. Frequency of administration of a dosage to a mammal can be twice per day. Thus, the amount of each inactive ingredient administered per day may be in a range of about 0.004 mg/kg/day to about 0.008 mg/kg/day. Dosage amounts for saw palmetto extract and aloe barbadensis extract alternatively may range from about 0.02 mg/kg to about 0.06 mg/kg. Frequency of administration of a dosage to a mammal can be twice per day. Thus, the amount of saw palmetto extract and aloe barbadensis extract administered per day may be in a range of about 0.04 mg/kg/day to about 0.12 mg/kg/day.

In a second embodiment, the present invention includes a pharmacologically acceptable composition for administration to a mammal including a first ingredient which may be growth hormone, or a substance to stimulate the production of growth hormone, and a second ingredient to synergistically enhance the effects of the first ingredient. Suitable first ingredients are natural or synthetic growth hormone, Mucuna Pruriens, or Tribulus L. Terrestris. The first ingredient may be present in homeopathic or non-homeopathic form. When the first ingredient is present in homeopathic form, the homeopathic first ingredient is present in the composition in an amount sufficient to increase levels of growth hormone within the human system. In various embodiments of the present invention, the first ingredient may be present in different homeopathic potencies. Particularly, the first ingredient may be provided in a potency of 10×. Alternatively, the potency may be 30×. Still alternatively, the potency may be 100×. These potencies may be combined in a 4 oz. (118 ml) solution. A dosage unit from this solution is in the range of about 0.03 oz. (0.9 ml) to about 0.08 oz. (2.4 ml). Particularly, a dosage unit may be about 0.07 oz. (2.1 ml). Frequency of administration of a dosage to a mammal can be twice per day.

Mucuna Pruriens contains L-dopa. The L-dopa naturally contained in the Mucuna Pruriens triggers the body to stimulate natural growth hormone production. The Mucuna Pruriens may be present in the composition of the present invention in an amount of at least 0.09 percent by volume. Mucuna Pruriens can be present in a range of about 50 mg to about 300 mg per 4 oz. (118 ml) solution. For example, 100 mg Mucuna Pruriens may be present in 4 oz. (118 ml) solution of the composition. For administration to a mammal, the dosage amount for Mucuna Pruriens may range from about 0.02 mg/kg to about 0.06 mg/kg. Frequency of administration of a dosage to a mammal can be twice per day. Thus, the amount of Mucuna Pruriens administered per day may be in a range of about 0.04 mg/kg/day to about 0.12 mg/kg/day.

The Tribulus L. Terrestris may be provided as an herb or herbal extract to stimulate the secretion of luteinizing hormones from the anterior pituitary gland, which triggers growth hormone production. The Tribulus L. Terrestris may be present in the composition of the present invention in an amount of at least 0.09 percent by volume. Tribulus L. Terrestris can be present in a range of about 50 mg to about 300 mg per 4 oz. (118 ml) solution. In one particular embodiment, for example, 100 mg of Tribulus L. Terrestris may be present in 4 oz. (118 ml) solution of the composition. For administration to a mammal, the dosage amount for Tribulus L. Terrestris may range from about 0.02 mg/kg to about 0.06 mg/kg. Frequency of administration of a dosage to a mammal can be twice per day. Thus, the amount of Tribulus L. Terrestris administered per day may be in a range of about 0.04 mg/kg/day to about 0.12 mg/kg/day.

A suitable second ingredient can be Morinda citrifolia (Noni), or an extract thereof. The first and second ingredients may be provided in homeopathic or non-homeopathic form. Morinda citrifolia is a fruit, the juice of which contains appreciable amounts of the precursor of an alkaloid referred to as xeronine. Xeronine is known to have an activity at the molecular level to repair damaged cells. Thus, Morinda citrifolia retards tumor growth by stimulating the immune system, is known to cause regeneration and increased cell function, is a natural antiseptic, and has an analgesic effect. The second ingredient may include an extract of Morinda citrifolia including xeronine. In particular, Morinda citrifolia may be present in an amount of 7 percent by volume. For administration to a mammal, the dosage amount for Morinda citrifolia may range from about 1.8 mg/kg to about 4.6 mg/kg. Frequency of administration of a dosage to a mammal can be twice per day. Thus, the amount of Morinda citrifolia administered per day may be in a range of about 3.6 mg/kg/day to about 9.2 mg/kg/day.

The composition including first and second ingredients, as described above, may also include a plurality of active homeopathic ingredients. These active homeopathic ingredients may create a synergistic effect of the composition which is more effective than using each ingredient individually. Thus, the composition includes a combination of growth hormone, or a substance to stimulate growth hormone, including Morinda citrifolia or an extract thereof, which may be delivered to the body along with a plurality of other active homeopathic ingredients, some of which may offer similar benefits that mimic growth hormone, and some of which may cause the body to produce more growth hormone by causing stimulation of the body's own growth hormone production.

The plurality of active homeopathic ingredients may include one or more of abrotanum, anacardium, orientate, arsenicum album, baryta carbonica, baryta muriatica, calcarea carbonica, calcarea fluorica, calcarea phosphorica, ferrum metallicum, fucus vesiculosus, hekla lava, helleborus niger, ignatia amara, lycopodium clavatum, nicotinamidium, secale cornutum, and silicea. Each of these active homeopathic ingredients may be present in concentrations of 10×. In an alternate embodiment, each of the active homeopathic ingredients may be present in concentrations of 30×. In yet another alternate embodiment, each of the active homeopathic ingredients may be present in concentrations of 100×. In one particular embodiment, potencies of 10×, 30×, and 100× of each ingredient are combined in a 4 oz. (118 ml) solution. A dosage unit from this solution is in the range of about 0.03 oz. (0.9 ml) to about 0.08 oz. (2.4 ml). Particularly, a dosage unit of this solution may be about 0.07 oz. (2.1 ml). Frequency of administration of a dosage to a mammal can be twice per day.

In addition to the ingredients listed above, the composition may include a plurality of inactive ingredients. In one embodiment, the plurality of inactive ingredients may include one or more of aloe barbadensis extract, polyacrylamide, C13-14 isoparaffin, laureth 7, lecithin, vitamin E acetate, sodium ascorbol phosphate, vitamin A, vitamin D3, vitamin B2, and diazolidinyl urea. The composition may include each of the above listed inactive ingredients. These inactive ingredients may be present in equal amounts, such as at least 0.01 percent by volume each, or, alternatively, may be present in varying amounts. For administration to a mammal, the dosage amount may range from about 0.002 mg/kg to about 0.004 mg/kg. Frequency of administration of a dosage to a mammal can be twice per day. Thus, the amount of each inactive ingredient administered per day may be in a range of about 0.004 mg/kg/day to about 0.008 mg/kg/day. Dosage amounts for aloe barbadensis extract may alternatively range from about 0.02 mg/kg to about 0.06 mg/kg. Frequency of administration of a dosage to a mammal can be twice per day. Thus, the amount of aloe barbadensis extract administered per day may be in a range of about 0.04 mg/kg/day to about 0.12 mg/kg/day.

Another aspect of the present invention reduces the cost of treatments and eliminates the need for physician supervision during treatment. In accordance with that aspect, the composition of the present invention may be formed into ingestable capsules, tablets, micro tablets or micro pellets by processes known in the art of pill manufacturing. Capsules may be formed by blending the component ingredients, such as herbal extracts, and subsequently filling capsules with the mixture using conventional automatic filling equipment. Tablets may be formed either by direct compression of components or by granulation followed by compression. Micro tablets may be formed by compressing powdered or granulated components into small diameter tablets. In one embodiment of the present invention, the composition is provided in capsule form.

The composition of the present invention may be provided in a transdermal gel utilizing liposome technology to transport the ingredients directly through the skin for maximum absorption. This eliminates the need for high cost injections and also eliminates oral supplements which are quickly broken down in the digestive tract of the human.

More specifically, in transdermal delivery form, the composition of the present invention may include a liposome as the vesicle. As known by those having skill in the art, liposomes are highly complex microscopic lipidspheres, ranging in size from 50 nanometers to several micrometers in diameter, and formed when phospholipids are hydrated. The phospholipids join “tails to tails” to build a bilayer membrane which may enclose water in a phospholipidsphere. By this structure, liposomes can encapsulate water soluble ingredients in their inner waterspace, and oil soluble ingredients in their phospholipid membranes. Liposomes may be made with concentrated phospholipids purified from natural lecithin and may be bioidentical to the phospholipids that make up cell membranes. The liposome used in the present invention may be unilamellar or multilamellar. Thus, the liposome may be unilamellar and the ingredients may be located within an interior chamber. Alternatively, the liposome may be multilamellar and having first and second interior chambers, with one or more of the ingredients being located in the first interior chamber and other of the ingredients being located in the second interior chamber. Still alternatively, the liposome may be multilamellar having first and second interior chambers, wherein certain of the ingredients are located in either the first or second interior chambers.

As described above, the vesicles for the gel or other substance for topical administration of the present invention include phospholipid membranes made of lipid components. More specifically, these vesicle-forming lipids may be an amphipathic lipid having a hydrophobic tail and a headgroup which conform spontaneously into bilayer vesicles in water. The vesicle-forming lipids are preferably ones having two hydrocarbon chains. These may be typically acyl chains, wherein the headgroup is either polar or nonpolar. As will be apparent to those having skill in the art, there are a variety of synthetic vesicle-forming lipids and naturally occurring vesicle-forming lipids suitable for use, such as phospholipids, which include, but are not limited to, phosphotidylcholine, phosphotidylethanolamine, phosphotidylserine, phosphotidylinositol, phosphotidic acid, and sphingomyelin. The two hydrocarbon chains may-be approximately 14-22 carbon atoms in length, and have varying degrees of saturation. Such lipids may be obtained commercially or prepared according to methods well known by those of skill in the art.

In addition to the vesicle-forming lipid component, the vesicles of the present invention can include other lipid components capable of being stably incorporated into lipid bilayers. These lipid bilayers have their hydrophobic moieties in contact with the interior, hydrophobic region of the bilayer membrane, and the polar headgroups oriented toward the exterior, polar surface of the membrane. For example, glycolipids, ceramides, and sterols, such as cholesterol, coprostanol, cholestanol, and cholestane, long-chain fatty acids, such as stearic acid, can be incorporated into the lipid bilayer. Other lipid components that may be used include fatty amines, fatty acylated proteins, fatty acylated peptides, oils, fatty alcohols, glyceride esters, petrolatum, and waxes. It will also be appreciated by those skilled in the art that a skin permeation enhancer may be included in lipid vesicle lipid components.

In preparing the composition of the present invention into a vesicle, the hormone, or hormone potentiating substance, active ingredients, inactive ingredients, and any other components of the composition, may be entrapped in the central core compartment of the vesicles, between the lipid bilayers, in the interior of the lipid bilayers.

Water soluble components may be entrapped in an interior compartment by adding those components to a water phase during preparation of an oil-in-water emulsion. The various ingredients of the composition, dissolved or suspended in the water phase, may be entrapped as part of the emulsion during lipid vesicle formation upon addition of the vesicle-forming lipids.

Lipophilic ingredients may be added to an oil phase during preparation of the oil-in-water emulsion for entrapment in the interior compartment. Alternatively, lipophilic ingredients may be entrapped in the lipid bilayer by adding the ingredients to the vesicle-forming lipid and/or the other lipid components, such as cholesterol.

### EXAMPLE

A pharmacologically acceptable composition for topical administration to a mammal for stimulating the increase of levels of testosterone within the mammalian system, was prepared as follows. Herbal extracts of Eurycoma longifolia jack, Tribulus, L. Terrestris, Mucuna Pruriens, Epimedium sagittatum, and Cnidium monnier were prepared. The herbs were first ground and then freeze dried. Extracts were then taken of the herbs such that the extract of Eurycoma longifolia jack included at least 20% glycoproteins and at least 40% glycosaponins; the extract of Tribulus L. Terrestris included at least 40% protodioscin; the extract of Mucuna Pruriens inlcuded at least 20% L-dopa; the extract of Epimedium sagittatum included at least 20% icariin; and the extract of Cnidium monnier included at least 50% osthole. The extracts were obtained through a low-temperature water extraction process and then tested by thin layer chromatography to analyze for the presence of any heavy metals or microbial toxins. 100 mg of each of these extracts was then liquefied. This liquefication occurs at room temperature and was performed by Cosmetic Concepts, Inc. of Swannanoa, N.C. Additionally, 100 mg of herbal extracts of each of Chrysin, Diindolylmethane, Aloe barbadensis and Saw palmetto were also liquefied. All the liquefied herbal extracts were then blended together in an industrial mixer.

Separately, a mixture of dilutions of various potencies of 12 active homeopathic ingredients was prepared. The mixture of active homeopathic ingredients included each of adrenalinium, alfalfa, avena sativa, baryta carbonica, baryta iodata, baryta muriatica, calcarea carbonica, calcarea fluorica, calcarea phosphorica, lycopodium clavatum, and thuja occidentalis, as active homeopathic ingredients. These active ingredients were each prepared in 3 separate potencies (10×, 30×, and 10×). To prepare the active homeopathic ingredients in the various potencies, each homeopathic ingredient was first obtained as an original tincture, which included 10 g of active ingredient dissolved in 90 g of solvent (to create a 10% solution). The solvent used was purified water. Next, the solution was repeatedly diluted. These dilutions were performed at room temperature. The dilution was repeated ten times to form a 10× potency; was repeated 30 times to form a 30× potency; and was repeated 100 times to form a 100× potency. The preparation of the active homeopathic ingredients was performed by King Bio Laboratories of Asheville, North Carolina. A 10× solution, a 30× solution, and a 100× solution, of each of the active homeopathic ingredients was then added to the liquefied and blended herbal extracts and mixed at room temperature in an industrial mixer. This mixing was performed by Cosmetic Concepts, Inc.

Additional ingredients, such as thickeners, emulsifiers, antioxidants, and anti-microbial agents were then added to the mixture. This also was performed by Cosmetic Concepts, Inc. Particularly, polyacrylamide and C13-14 isoparaffin were added as thickeners; laureth 7 was added as an emulsifier; diazolidinyl urea was added as an anti-microbial agent; and disodium EDTA was added as an antioxidant, chelating agent, and thickener. Further, lecithin was added to the mixture. Lecithin is present for the preparation of liposomes, in which the other ingredients will be contained, for topical administration and transdermal absorption into a body. Particularly, the mixture prepared included varying amounts of each of the above-listed additional ingredients. In particular, the mixture included:

| Ingredient | Amount |
|---|---|
| Eurycoma longifolia jack (extract) | 100 mg |
| Tribulus L. Terrestris (extract) | 100 mg |
| Mucuna Pruriens (extract) | 100 mg |
| Epimedium sagittatum (extract) | 100 mg |
| Cnidium monnier (extract) | 100 mg |
| Chrysin (extract) | 100 mg |
| Diindolylmethane (extract) | 100 mg |
| Aloe barbadensis (extract) | 100 mg |
| Saw palmetto (extract) | 100 mg |
| Adrenalinium | 10X, 30X, 100X |
| Alfalfa | 10X, 30X, 100X |
| Avena sativa | 10X, 30X, 100X |
| Baryta carbonica | 10X, 30X, 100X |
| Baryta iodata | 10X, 30X, 100X |
| Baryta muriatica | 10X, 30X, 100X |
| Calcarea carbonica | 10X, 30X, 100X |
| Calcarea fluorica | 10X, 30X, 100X |
| Calcarea phosphorica | 10X, 30X, 100X |
| Lycopodium clavatum | 10X, 30X, 100X |
| Thuja occidentalis | 10X, 30X, 100X |
| Polyacrylamide | 5.5 mg (<0.5% of total) |
| C13-14 isoparaffin | 5.5 mg (<0.5% of total) |
| Laureth 7 | 5.5 mg (<0.5% of total) |
| Lecithin | 5.5 mg (<0.5% of total) |
| Disodium EDTA | 5.5 mg (<0.5% of total) |
| Diazolidinyl urea | 5.5 mg (<0.5% of total) |

The above mixture was then encapsulated in liposomes as described in the Detailed Description by methods known to those having skill in the art. Such methods were particularly performed by Cosmetic Concepts, Inc. In particular, the above mixture was encapsulated into liposomes in a total 4 oz (118 ml) solution. Such a 4oz solution contains sixty dosage units, each dosage unit being 0.067 oz.

While the present invention has been illustrated by a description of various embodiments and while these embodiments have been described in considerable detail, it is not the intention of the applicants to restrict or in any way limit the scope of the appended claims to such detail. Additional advantages and modifications will readily appear to those skilled in the art. The invention in its broader aspects is therefore not limited to the specific details, and representative composition and method shown and described. Accordingly, departures may be made from such details without departing from the spirit or scope of applicant's general inventive concept.

What is claimed is:

1. A pharmaceutically acceptable composition for administration to a mammal, comprising:
a first ingredient chosen from a hormone, a composition which potentiates a hormone, and mixtures thereof; and
a second ingredient effective to stimulate the production of cyclic Gmp.

2. The composition of claim 1, wherein said hormone is testosterone.

3. The composition of claim 2, wherein said first ingredient potentiates luteinizing hormone.

4. The composition of claim 3, wherein said first ingredient is Eurycoma longifolia jack.

5. The composition of claim 4, wherein said Eurycoma longifolia jack is present in said composition in a dosage amount in a range of about 0.02 mg/kg to about 0.06 mg/kg.

6. The composition of claim 3, wherein said first ingredient is Tribulus L. Terrestris.

7. The composition of claim 6, wherein said Tribulus L. Terrestris is present in said composition in a dosage amount of about 0.02 mg/kg to about 0.06 mg/kg.

8. The composition of claim 1, wherein said second ingredient includes a coumarin.

9. The composition of claim 8, wherein said coumarin stimulates the production of nitric oxide.

10. The composition of claim 9, wherein said coumarin is osthole.

11. The composition of claim 10, wherein said second ingredient is Cnidium monnier.

12. The composition of claim 11, wherein said Cnidium monnier is present in said composition in a dosage amount in a range of about 0.02 mg/kg to about 0.06 mg/kg.

13. The composition of claim 1, wherein said second ingredient inhibits the activity of at least one enzyme.

14. The composition of claim 13, wherein said enzyme is a phophodiesterase.

15. The composition of claim 14, wherein said enzyme is phophodiesterase-5.

16. The composition of claim 15, wherein said second ingredient is Cnidium monnier.

17. The composition of claim 1, further comprising a third ingredient for stimulating an increase in blood flow.

18. The composition of claim 17, wherein said third ingredient is Epimedium sagittatum.

19. The composition of claim 18, wherein said Epimedium sagittatum is present in said composition in a dosage amount in a range of about 0.02 mg/kg to about 0.06 mg/kg.

20. The composition of claim 1, wherein said first ingredient is provided in homeopathic form.

21. The composition of claim 1, wherein said second ingredient is provided in homeopathic form.

22. The composition of claim 17, wherein said third ingredient is provided in homeopathic form.

23. The composition of claim 1, wherein said first ingredient and said second ingredient are provided in a capsule for ingestion into a body.

24. The composition of claim 1, further comprising at least one vesicle operable for transporting said first ingredient and said second ingredient from a first site external to a body to a second site internal to said body.

25. The composition of claim 24, wherein said at least one vesicle is a liposome having an interior chamber.

26. The composition of claim 25, wherein said liposome is formed from phopholipids selected from the group consisting of phosphotidylcholine, phosphotidylethanolamine, phosphotidylserine, phosphotidylinositol, phophotidic acid, and sphingomyelin.

27. The composition of claim 1, further including a plurality of active homeopathic ingredients.

28. The composition of claim 27, wherein the active homeopathic ingredients are chosen from abrotanum, adrenalinum, alfalfa, anacardium orientale, arsenicum album, avena sativa, baryta carbonica, baryta iodata, baryta muriatica, calcarea carbonica, calcarea fluorica, calcarea phosphorica, ferrum metallicum, fucus vesiculosus, hekla lava, helleborus niger, ignatia amara, lycopodium clavatum, nicotinamidium, secale cornutum, silicea, or thuja occidentalis.

29. The composition of claim 1, further including a plurality of inactive ingredients.

30. The composition of claim 29, wherein said inactive ingredients are chosen from epimedium extract, aloe barbadensis extract, polyacrylamide, C13-14 isoparaffin, indole-3-carbinol, laureth 7, lecithin, saw palmetto extract, diazolidinyl urea, vitamin E acetate, sodium ascorbol phosphate, vitamin A, vitamin D3, or vitamin B2.

31. A pharmaceutically acceptable composition for administration to a mammal, comprising:
a first ingredient chosen from testosterone, a substance to potentiate testosterone, and mixtures thereof; and
a second ingredient effective to stimulate the production of cyclic GMP.

32. A pharmaceutically acceptable composition for topical administration to a mammal, comprising:
a first ingredient effective to stimulate the synthesis of cyclic GMP; and
at least one vesicle operable for transporting said first ingredient from a first site external to a body to a second site internal to said body.

33. The composition of claim 32, wherein said first ingredient includes a coumarin.

34. The composition of claim 32, wherein said first ingredient stimulates the production of nitric oxide.

35. The composition of claim 32, wherein said first ingredient inhibits the activity of at least one enzyme.

36. The composition of claim 32, wherein said first ingredient is Cnidium monnier.

37. A pharmaceutically acceptable composition for administration to a mammal, comprising:
a first ingredient chosen from a hormone, a composition which potentiates a hormone, and mixtures thereof; and
a second ingredient chosen from Morinda citrifolia and an extract of Morinda citrifolia.

38. The composition of claim 37, wherein said first ingredient is growth hormone.

39. The composition of claim 37, further including a third ingredient including a luteinizing agent for stimulating the production of a hormone by a body.

40. The composition of claim 39, wherein said third ingredient is chosen from Mucuna Pruriens and Tribulus L. Terrestris.

41. The composition of claim 37, further including a plurality of active homeopathic ingredients.

42. The composition of claim 41, wherein the active homeopathic ingredients are chosen from abrotanum, adrenalinum, alfalfa, anacardium orientate, arsenicum album, avena sativa, baryta carbonica, baryta iodata, baryta muriatica, calcarea carbonica, calcarea fluorica, calcarea phosphorica, ferrum metallicum, fucus vesiculosus, hekla lava, helleborus niger, ignatia amara, lycopodium clavatum, nicotinamidium, secale cornutum, silicea, or thuja occidentalis.

43. The composition of claim 37, further including a plurality of inactive ingredients.

44. The composition of claim 43, wherein said inactive ingredients are chosen from epimedium extract, aloe barbadensis extract, polyacrylamide, C13-14 isoparaffin, laureth 7, lecithin, saw palmetto extract, diazolidinyl urea, vitamin E acetate, sodium ascorbol phosphate, vitamin A, vitamin D3, or vitamin B2.

45. The composition of claim 37, further including at least one vesicle operable for transdermally transporting said first ingredient and said second ingredient from a first site external to a body to a second site internal to said body.

46. The composition of claim 37 wherein said first ingredient includes an herbal extract including an element which synthesizes a catecholamine.

47. The composition of claim 46 wherein said element of said herbal extract is a hydroxylated amino acid.

48. The composition of claim 47 wherein said hydroxylated amino acid is L-dopa.

49. The composition of claim 46 wherein said catecholamine to be synthesized is dopamine.

50. The composition of claim 48 wherein said herbal extract is an extract of Mucuna Pruriens.

51. The composition of claim 46, wherein said first ingredient includes an herbal extract having an active component comprising a luteinizing agent.

52. The composition of claim 51, wherein said herbal extract is an extract of Tribulus L. Terrestris.

53. The composition of claim 46, wherein said element for synthesizing a catecholamine is L-dopa, said L-dopa operable to stimulate said mammal to synthesize dopamine.

54. The composition of claim 53, further comprising a third ingredient operable to prevent L-dopa from degrading in a mammal, thereby enhancing dopamine uptake in the mammal.

55. The composition of claim 54, wherein said third ingredient is Tribulus L. Terrestris or an herbal extract thereof.

56. The composition of claim 46, further including a plurality of active homeopathic ingredients.

57. The composition of claim 56, wherein the active homeopathic ingredients are chosen from abrotanum, adrenalinum, alfalfa, anacardium orientale, arsenicum album, avena sativa, baryta carbonica, baryta iodata, baryta muriatica, calcarea carbonica, calcarea fluorica, calcarea phosphorica, ferrum metallicum, fucus vesiculosus, hekla lava, helleborus niger, ignatia amara, lycopodium clavatum, nicotinamidium, secale cornutum, silicea, or thuja occidentalis.

58. The composition of claim 46, further including a plurality of inactive ingredients.

59. The composition of claim 58, wherein said inactive ingredients are chosen from epimedium extract, aloe barbadensis extract, polyacrylamide, C13-14 isoparaffin, laureth 7, lecithin, saw palmetto extract, diazolidinyl urea, vitamin E acetate, sodium ascorbol phosphate, vitamin A, vitamin D3, or vitamin B2.

60. The composition of claim 46, further including at least one vesicle operable for transdermally transporting said first ingredient and said second ingredient from a first site external to a body to a second site internal to said body.
